(19) United States
(12) Patent Application Publication
(10) Pub. No.: US 2004/0157876 A1
(43) Pub. Date: Aug. 12, 2004

(54) Granular preparations of gaboxadol

(76) Inventors: Michiel O Elema, Kobenhavn (DK); Lene Andresen, Malov (DK); Per Holm, Vanlose (DK)

Correspondence Address: DARBY & DARBY P.C., P. O. BOX 5257, NEW YORK, NY, 10150-5257, US

(21) Appl. No.: 10/478,983

(22) PCT Filed: May 17, 2002
(86) PCT No.: PCT/DK02/00332

(30) Foreign Application Priority Data
May 21, 2001 (DK) PA200100817

(51) Int. Cl. A61K 31/4745

(52) U.S. Cl. 514/302

(57) ABSTRACT

The present invention relates to a granulated product containing gaboxadol as an acid addition salt, a melt granulation process for the preparation thereof and to solid pharmaceutical unit dosage forms prepared from said granular preparation of gaboxadol.

The present invention relates to a granulated product containing gaboxadol, a melt granulation process for the preparation thereof and to solid pharmaceutical unit dosage forms prepared from said granular product.

### BACKGROUND

Solid, shaped pharmaceutical unit dosage forms, such as tablets, are prepared by compression of the dry ingredients, which are in the form of powders or small particles. The methods and excipients used for the compression of tablets are well known in the art. The choice of pharmaceutical excipients for a particular formulation largely depends on the physico/chemical properties including the tabletting properties of the active ingredient.

Reproducible dosing for tabletting requires that all the dry ingredients have good fluidity properties. In some cases, where the active ingredient has good fluidity properties, tablets can be prepared by direct compression of the ingredients. However, in many cases, where the particle size of the active substance is small, the active substance will be cohesive and have poor fluidity properties. To ensure optimal flowability and to ensure homogenous mixture of compounds, agglomerates of active compound and excipients are prepared, usually by granulation of the active ingredient either alone or in combination with a filler or other conventional tablet ingredient.

One such granulation method is the “wet” granulation process. Using this method, the dry solids (active ingredients, binder etc.) are blended and moistened with water or another wetting agent (e.g. an alcohol) and agglomerates or granules are build up of the moistened solids. Blending is continued until a desired homogenous particle size has been achieved whereafter the granulated product is dried.

The “wet” granulation process is widely employed for the granulation of powders or fine particles where water can be used as the wetting agent. The compound, gaboxadol, which has the formula:

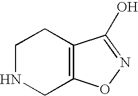

is a valuable hypnotic. The compound is considered of particular interest for the treatment of sleep disorders (U.S. Pat. No. 5,929,065). In the case of the active ingredient being gaboxadol as an acidic reacting addition salt, in this particular instance the hydrochloric acid salt, the technique of wet granulation presented a number of problems. Analogous problems could be expected for other acidic reacting salts such as for example the hydrobromic acid salt.

Tabletting with a wet granulation of gaboxadol, HCl consisting of maize starch, lactose, croscarmellose sodium and hydroxypropylcellulose can be performed without technical problems. However, observations were made, that corrosion of the tabletting equipment occurred while working with the product. Several parts of the tabletting equipment are made of steel and iron and corrosion of these parts could also be detected after the granulate had been in contact with the equipment for several hours. During this corrosion process, iron(III) ions are released from the equipment due to the low pH of the aqueous solution of gaboxadol, HCl. Possibly, gaboxadol is also able to form complexes with the released iron(III), which could be coloured.

As most tabletting equipment has iron containing parts, it is not possible to completely avoid iron in the tabletting process.

A solution to the above problem is the manufacturing process described in the present invention. Water is avoided by using anhydrous excipients and a melt granulation using a non-aqueous binder.

### OBJECT OF THE INVENTION

It is the object of the present invention to provide a granular preparation containing gaboxadol, HCl which can be used for the preparation of solid, shaped pharmaceutical unit dosage forms containing gaboxodol, HCl which are stable upon storage. It is also an object of the invention to provide a granular preparation of gaboxadol, HCl which has a suitable release profile.

### SUMMARY OF THE INVENTION

The invention then inter alia comprises the following alone or in combination:

A granulated product containing the active ingredient, gaboxadol, and besides the active ingredient, which may be in the form of a granulated product. Solid, pharmaceutical unit dosage forms usually include various other conventional excipients such as additional fillers, binders, disintegrants, and optionally minor amounts of lubricants, colorants and sweeteners.

The choice of the excipients largely depends on the physico/chemical properties of the active ingredient, including the tabletting properties of the active ingredients and the stability of the final composition.

Suitable fillers for the preparation of solid, unit dosage forms according to the invention include sugars (sorbitol, mannitol, dextrose, sucrose), lactose, calcium phosphates, starch, maize starch, modified starches, microcrystalline cellulose, calcium sulphate, calcium carbonate. The fillers should be anhydrous and preferably non-hygroscopic.

In a preferred embodiment of the invention, maize starch or calcium phosphates are used or a combination of maize starch and calcium phosphates.

The filler may be added to or mixed with the granulated product after granulation or it can be granulated together with the active ingredient or both.

Disintegrants include sodium starch glycolate, croscarmellose sodium, crospovidone, low substituted hydroxypropylcellulose, modified cornstarch, pregelatizined starch and natural starch.

Examples of lubricants include metallic stearates (magnesium, calcium, sodium), stearic acid, wax, hydrogenated vegetable oil, talc, colloidal silica and sodium benzoate.

Preferably, the mentioned excipients are anhydrous and non-hygroscopic.

A melt granulated product containing

a) 5-40% of a hydrophilic melt binder

b) 0-90% filler, and

c) gaboxadol as the free base, as the hydrate or with a pharmaceutically acceptable acid addition salt thereof.

Suitably, the melt granulated product contains 50-90% filler.

Suitable fillers for the granulated product include sugars (sorbitol, mannitol, dextrose, sucrose), calcium phosphates (dibasic, tribasic and anhydrous), starch, modified starches, microcrystalline cellulose, calcium sulfate and calcium carbonate.

In a preferred embodiment of the invention, the filler granulated together with the pharmaceutically acceptable salt of gaboxadol is anhydrous calcium phosphate. In another preferred embodiment, the filler is a mixture of anhydrous calcium phosphate and maize starch.

Suitably, the hydrophilic melt binder is added in an amount from 5 to 30%, or from 10 to 20%, or more preferred in an amount around 10-15%. Most preferred is hydrophilic melt binder in an amount of 10-12%, when the filler is CaHPO₄.

In one embodiment of the invention, the hydrophilic melt binder is a polyethylene glycol of the formula HO—(CH₂CH₂O)ₙ—H, which is available with various average molecular weights. PEG having an average molecular weight from 1000 to 10000 is suitable for the preparation of the granular product according to the invention. PEG 3000 (PEG with an average molecular weight around 3000) has a melting range 48-54° C.; PEG 4000 has a melting range around 50-58° C., PEG 6000 has a melting range around 55-63° C. and PEG 8000 has a melting range around 60-63° C.

Other polyether glycols such as polypropylene glycol, polyethylene glycol esters or acids, as well as polyoxypropylene and polyethylene oxide and copolymers thereof may also be used as the hydrophilic melt binder.

In a preferred embodiment of the invention, the melt binder used is PEG 6000.

The active ingredient is present in the granulated product in a suitably amount, which is up to 50% of the granulated product. In preferred embodiments of the present invention, the amount is below 30%, and more preferred between 2 and 25%. In the most preferred embodiment of the invention, the amount is between 3 and 10%. All of the above procentages are calculated from the active compound and as used herein, % means %(w/w).

The invention also relates to a method for the preparation of a granulated product containing a pharmaceutically acceptable salt of gaboxadol which comprises blending of the dry ingredients while heating to a temperature above the melting point of the hydrophilic melt binder, followed by mechanical working until a uniform granular product is formed. The ingredients are preferably granulated in one step starting with the total amount of all ingredients. Lubricants, if present, are added immediately before the tabletting process.

Where the granulating agent is PEG 6000, a suitable temperature for the granulation process is between 60-85° C. The granulation process may be carried out in a jacketed bowl equipped with blending means, in fluidised bed or any other apparatus suitable for carrying out granulation provided heat can be induced.

The granulating agent is dry-blended with the other ingredients (i.e. active ingredient and filler) prior to heating. Alternatively, the granulating agent is melted and continuously added to or sprayed on an agitated mixture of the other ingredients.

The granulation mixture is heated to substantially liquefy the granulating agent, and thereafter heated and mechanically worked or agitated until the desired particle size is achieved. The granulated product is cooled to a temperature below the melting point of the granulating agent. The granulated product may be continuously agitated or worked throughout the heating and the cooling phase in order to obtain a homogenous granulate.

As an alternative, granulation can be carried out in fluid bed equipment. Using this technique, the melted granulating agent is added to the fluidised bed of the other components. In a special embodiment of this technique, the granulating agent is sprayed into the fluid bed. Fluid bed melt granulation can also be carried out as described in DE 21 27 683.

In a final embodiment, the invention comprises a composition containing the melt granulated product containing gaboxadol together with conventional pharmaceutical excipients.

In a preferred embodiment of the invention, the composition according to the invention is in the form of a solid, shaped pharmaceutical unit dosage form, i.e. a tablet. In one embodiment of the invention, the tablets are prepared by direct compression.

The solid and shaped pharmaceutical unit dosage forms may be prepared by conventional methods and apparatus for the compression of tablets.

The pharmaceutical unit dosage forms may optionally be coated by techniques known in the art and with coating agents also known in the art. Good results were obtained with commercially available film coating suspensions.

In the following, the invention is illustrated by way of examples. However, the examples are merely intended to illustrate the invention and should not be construed as limiting.

### EXAMPLE 1

### Preparation of a melt granulated product containing gaboxadol, HCl as the active ingredient and compression of 200 mg tablets containing 5 mg active ingredient.

| Ingredients for granulation: | | |
|---|---|---|
| Active ingredient | 15.75 g | (3.15%) |
| Polyethylene glycol 6000 | 58.4 g | (11.68%) |
| Calcium hydrogen phosphate anhydrous | 412.4 g | (82.47%) |

Melt granulation in fluid-bed:

The in-let air temperature of the fluid-bed was set to 90° C. Calcium hydrogen phosphate anhydrous was combined with gaboxadol and PEG 6000 in the fluid-bed and blended. The process was continued after the melting point of PEG for 3-5 minutes, while the temperature was allowed to rise to a temperature between 65-80° C. The granulated product was cooled and passed through a 1 mm mesh screen.

Melt granulation in high shear mixer:

The temperature regulator of a heat jacketed high shear mixer was set to 80° C. Calcium hydrogen phosphate anhydrous was combined with gaboxadol and PEG 6000 in the mixer and blended at 1200 rpm until peak power consumption of the motor was measured. Blending was continued at 800 rpm for 2-4 minutes while the temperature was allowed to rise to a temperature between 60-75° C. The granulated product was cooled and passed through a 1 mm mesh screen.

Screen analysis

Geometric weight mean diameter (dgw): 100-250 μm

Geometric standard deviation (Sg): 2-3

| Tablet ingredients: | | |
|---|---|---|
| Melt granulate | 500 g | (97.3%) |
| Croscarmellose sodium | 10.3 g | (2%) |
| Magnesium stearate | 3.6 g | (0.7%) |

Magnesium stearate was passed through a 0.2 mm mesh screen. The gaboxadol melt-granulate and croscarmellose sodium were blended. Magnesium stearate was added and blended. The resulting composition was loaded into a Korch PH 106 tabletting machine mounted with oval 5.5×8 mm punches and pressed into tablets with a core weight of 200 mg.

### EXAMPLE 2

Analogous to the above, the following experiments were performed:

| | |
|---|---|
| Active ingredient | 5% |
| PEG 6000 | 14.6% |
| Maize starch | 77.7% |
| Croscarmellose Sodium | 2% |
| Magnesium stearate | 0.7% |

### EXAMPLE 3

| | |
|---|---|
| Active ingredient | 4.2% |
| PEG 6000 | 10.5% |
| CaHPO₄ anhydrous | 30.3% |
| Maize Starch | 30.3% |
| Microcrystalline cellulose | 20% |
| Sodium Starch glycollate | 4% |
| Magnesium Stearate | 0.7% |

Experiments on the tablets formed by the above procedures have shown that no corrosion on the equipment was observed and that the tablets were very stable upon storage. Likewise, the dissolution time of the tablets is satisfactory.

1. A melt granulated product containing gaboxadol as a free base, as the hydrate or as a pharmaceutically acceptable acid addition salt thereof and excipients and fillers.

2. The granulated product according to claim 1, wherein the pharmaceutically acceptable salt of gaboxadol is the HCl salt.

3. The granulated product according to any of the claims 1 to 2, wherein the filler predominantly consists of CaHPO₄.

4. The granulated product according to any of the claims 1 to 2, wherein the filler predominantly consist of maize starch.

5. The granulated product according to any of the claims 1 to 2, wherein the filler is a combination of maize starch and CaHPO₄.

6. The granulated product according to any of the claims 1 to 5, wherein the granulation is performed in the presence of polyethylenglycol.

7. The granulated product according to claim 6, wherein the hydrophilic melt binder is polyethylene glycol having an average molecular weight of about 1000 to 10000.

8. The granulated product according to any of the claims 6 to 7, wherein the hydrophilic melt binder is polyethylene glycol having an average molecular weight of about 3000 to 8000.

9. The granulated product according to any of the claims 6 to 8, wherein the hydrophilic melt binder is PEG 6000.

10. The granulated product according to any of the claims 6 to 9, wherein the content of PEG is from 10-25%.

11. A process for the preparation of a granulated product according to any of the claims 1 to 10 comprising heating and mechanically working of a mixture containing
a) a hydrophilic binder having a melting point between 40° C. and 100° C.
b) 0-90% filler and
c) a pharmaceutically acceptable salt of gaboxadol as the free base, as the hydrate or a as a pharmaceutically acceptable addition salt thereof,
to a temperature above the melting point of the hydrophilic melt binder, until a homogenous granular product is formed.

12. The process of claim 11, wherein the melting temperature of the hydrophilic binder is between 60° C. and 85° C.

13. A melt granulated product according to any of the claims 1 to 12, obtainable by heating and mechanical working of a mixture containing
a) a hydrophilic binder having a melting point between 40° C. and 100° C.
b) 0-90% filler and
c) gaboxadol as the free base, as the hydrate or a as a pharmaceutically acceptable addition salt thereof,
to a temperature above the melting point of the hydrophilic melt binder, until a homogenous granular product is formed.

14. The melt granulated product according to claim 13, obtainable by heating and mechanical working of mixture containing a hydrophilic binder having a melting point between 60° C. and 85° C.

15. A composition comprising a melt granulated product according to any of the claims 1 to 10 or 13 to 14 together with conventional pharmaceutical excipients.

16. A composition according to claim 15 which is in the form of a solid, shaped pharmaceutical unit dosage form.

17. A solid, shaped pharmceutical unit dosage form according to claim 16 which is coated.
